(19) United States
(12) Patent Application Publication
(10) Pub. No.: US 2021/0391075 A1
(43) Pub. Date: Dec. 16, 2021

(54) Medical Literature Recommender Based on Patient Health Information and User Feedback

(71) Applicant: American Medical Association, Chicago, IL (US)

(72) Inventors: Joseph William Marks, Watertown, MA (US); Daniel Pickhardt, Chicago, IL (US); William Paul Gee, Chicago, IL (US); Amber Raschel Aurora Brown, Pittsburgh, PA (US); Lucia deFatima Soares, San Jose, CA (US); Roel Carl Nuyts, San Francisco, CA (US)

(21) Appl. No.: 16/900,751

(22) Filed: Jun. 12, 2020

(51) Int. Cl. G16H 50/20; G16H 50/70; G16H 70/20; G16H 15/00; G16H 10/60; G06Q 30/02; G16H 70/60; G16H 10/40; G06F 16/9538; G06F 16/9532; G06F 16/9535; G06F 16/28; G06N 5/04; G06F 40/20

(52) U.S. Cl. CPC G16H 50/20; G16H 50/70; G16H 70/20; G16H 15/00; G16H 10/60; G06Q 30/0282; G06F 40/20; G16H 10/40; G06F 16/9538; G06F 16/9532; G06F 16/9535; G06F 16/285; G06N 5/04; G16H 70/60

(57) ABSTRACT

A system recommends to a healthcare professional (HCP) (230) medical literature that is of relevance to the HCP's patients. The system communicates with the HCP (230) and accesses electronic health record (EHR) documents in a database (210) associated with the HCP's patients. The system analyzes the contents of the EHR documents to query a medical-literature database (212) for publications that are deemed relevant to the EHR documents. The extracted publications are then presented to the HCP.

### FIELD OF THE INVENTION

The present disclosure generally relates to a recommender system for obtaining and presenting documents from a document database, and more particularly to a computer-based recommender system that recommends medical-literature documents to healthcare professionals based on a computer processing of their patients' electronic health records.

### BACKGROUND

Document databases containing medical literature, including publications, articles, guidelines, tutorials, and the like, can be a useful resource for a healthcare professional (HCP). In some instances, finding a particular document may be as easy as submitting a search query to a search engine and receiving search results. For example, if the documents that form a corpus of documents are stored in a document repository along with indexes to the corpus, and those indexes include author, title, keywords, publication date, etc., an HCP may simply enter a name of a known author and a known title. However, often, the HCP is searching for something not so easily identified, or the HCP may have an interest in some document where the nature of the document is not precisely known, such as a reference to a prior example of a medical condition the HCP rarely encounters.

As one example, MEDLINE, a bibliographic database of the U.S. National Library of Medicine, could constitute such a healthcare document corpus. More than one million citations are added annually to just this one medical-literature database. Such a volume of publications can be overwhelming. Consequently, it can be very difficult for an HCP to stay informed about research that is relevant to the HCP's patients. There is therefore a need for systems to help make the growing medical literature more accessible to HCPs.

A system for accessing the MEDLINE medical-literature corpus, or at least references and abstracts to the articles therein, is PubMed, a search engine open to free public access. Simple searches on PubMed can be carried out by entering search terms into PubMed's search window. PubMed expands these initial search terms by automatically adding field names in which to search, relevant MeSH (Medical Subject Headings) terms, synonyms, Boolean operators, and organizing the exploded terms appropriately, thereby enhancing the original search query significantly. In addition to specifying keywords, the system also allows the user to filter results by publication type (e.g., journal article, clinical trial, guidelines, etc.), text availability, publication dates, languages, gender, species, etc.

In medical informatics, common barriers to effective and efficient searching of such data might include a failure to begin with a well-built clinical question, a failure to leverage the indexing system, failure to exploit important relationships between recall and precision, and failure to apply proper limits to a search. In particular, to make the most effective use of a search engine like PubMed, an HCP may need considerable skill to know which search terms will be most effective, have some notion of how the search terms will be expanded into the actual database queries, and be familiar with the often-arcane characteristics and categories of the database being queried. These failures often manifest themselves in retrieving too few or too many useful publications.

Because of the inherent challenges in using PubMed effectively, commercial vendors have therefore developed hundreds of alternative interfaces to PubMed for accessing MEDLINE and similar databases. These alternative interfaces differ in how search queries may be expressed and in how results are presented (e.g., the results might be rank ordered or visually clustered in intuitive ways). But in most of these systems the HCP is still responsible for the onerous tasks of specifying search terms and of interpreting the presented results.

In summary, an improved system for accessing the documents in a document corpus and identifying recommended references therein is needed, especially in the field of medicine.

### SUMMARY

A recommender system delivers a selection of relevant publications from the medical literature (e.g., medical journal articles, clinical studies, guidelines, presentations, videos, podcasts, blog postings, etc.) to a user such as a healthcare professional (HCP), patient, or patient caregiver. The selection of publications is relevant to the HCP user because it may be based in part on information extracted from a database of the HCP's patients' electronic health records (EHRs). The recommender system may also be influenced in part by user feedback concerning the relevance and utility of recommended publications. Some embodiments might include search features.

An embodiment system might comprise a network interface, a computing system, and at least one computing device configured to implement one or more services, wherein the one or more services are configured to access, over a network using the network interface, a set of EHRs relating to a set of patients from at least a first data store, use a first set of AI techniques to analyze the contents of the set of EHRs to extract a set of extracted medical facts, use a second set of AI techniques to formulate a set of database queries based on the set of extracted medical facts that are used to retrieve, over a network using the network interface, a set of resource locators, each resource locator for a retrieved medical-literature publication from at least a second data store that are relevant to the set of extracted medical facts, use a third set of AI techniques to determine a subset of the set of resource locators to present to a user, and present the subset of the set of the resource locators to a user via the computing system's display.

The embodiment might further provide that the first set of AI techniques analyzes the contents of the set of EHRs to extract a set of extracted medical facts using natural language processing (NLP).

The embodiment might further provide that the second set of AI techniques used to formulate a set of database queries based on the set of medical facts that are used to retrieve, over a network using the network interface, a set of retrieved medical-literature publications from at least a second data store that are relevant to the set of extracted medical facts, comprise one or more of these methods: expert-system rules that consider the diagnoses of one or more patients in the set of patients, expert-system rules that consider the therapies of one or more patients in the set of patients, expert-system rules that consider the comorbidities of one or more patients in the set of patients, expert-system rules that consider adjuvant or alternative therapies for one or more patients in the set of patients, and expert-system rules that consider the genomic profiles of one or more patients in the set of patients.

The embodiment might further provide that the third set of AI techniques used to determine which subset of the set of retrieved medical-literature publications to present to a user and how to present them, comprise one or more of these methods: term-matching and term-weighting methods to rate the relevance of publications in the set of retrieved medical-literature publications, expert-system rules that consider the citation counts or download counts of publications in the set of retrieved medical-literature, expert-system rules that consider user-supplied feedback regarding the publications in the set of retrieved medical-literature publications, expert-system rules that promote variety in the subset of the set of retrieved medical-literature publications to present to a user, expert-system rules that apply pedagogical strategies in the selection of publications in the subset of the set of retrieved medical-literature publications to present to a user, the provision of automatically generated explanations associated with the subset of the set of retrieved medical-literature publications to present to a user, and document-clustering methods for visually organizing and presenting the subset of the set of retrieved medical-literature publications to a user.

The following detailed description together with the accompanying drawings will provide a better understanding of the nature and advantages provided.

### BRIEF DESCRIPTION OF THE DRAWINGS

Various embodiments in accordance with the present disclosure will be described with reference to the drawings, in which:

FIG. 1 illustrates a presentation of document icons that uses document clustering and the concept of representative documents that are indicative of their clusters.

FIG. 2 depicts a system for recommending medical literature of relevance to patients of a healthcare professional.

FIG. 3 shows a sample free-text note from an electronic healthcare record (EHR).

FIG. 4 is a flowchart for an EHR fact-extraction module.

FIG. 5 illustrates expert-system rules for medical-literature query formulation.

FIG. 6 illustrates expert-system presentation rules.

FIG. 7 depicts a screenshot of an input/output module, with pulldown menus shown.

FIG. 8 depicts a screenshot of an input/output module, with a publication selected.

FIG. 9 depicts a screenshot of an input/output module, with a user-feedback dialog selected.

FIG. 10 illustrates an exemplary computer system for executing an embodiment.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Disclosed herein is a recommender system for delivering to a healthcare professional (HCP) a selection of relevant documents from the medical literature. The relevance of the publications from the medical literature might be measured against the electronic health records (EHRs) for the HCP's patients. This might result in the publications speaking to the conditions and therapies described in the EHRs. As used herein, a collection of electronically available or electronically referenced documents might be referred to as a document corpus.

Medical literature might be organized as several publication corpuses. The documents in a document corpus might be accessed via one or more database search engines, which use one or more content indices to organize and retrieve documents that meet certain search criteria, such as the inclusion of key words from a search query in the title, abstract, or body in the documents retrieved. A database search engine may return hits in the form of copies of medical-literature documents in various document formats, abstracts, metadata, links, references, or some other publication identifiers. The hits returned by one or more database search engines are ordered and organized before being returned to the HCP user of the recommender system. In the healthcare context, the document corpus might include medical journal articles, clinical studies, guidelines, presentations, videos, podcasts, blog postings, and the like.

As used herein, HCPs might include individuals who have completed a course, training program, or degree program in the medical profession, who have been licensed by a governmental agency, and/or who have been certified or approved by a medical professional organization to provide medical care. Example HCPs include, but are not limited to, physicians, nurses, pharmacists, nursing assistants, dental assistants, dentists, medical laboratory technicians, respiratory therapists, occupational therapists, physical therapists, etc. The recommender system disclosed herein could also be adapted to deliver a selection of relevant publications from the medical literature to patients and their caregivers.

The medical literature is the collective term for medical research documents archived in various libraries and repositories, collectively referred to as document corpuses. Examples of medical-research publications include journal articles, clinical studies, guidelines, presentations, videos, podcasts, blog postings, and the like. One such document corpus is MEDLINE, the U.S. National Library of Medicine bibliographic database that contains more than 25 million references to documents in the life sciences, with a concentration on biomedicine. A distinctive feature of MEDLINE is that its records are indexed with MeSH, or Medical Subject Heading, terms to enable effective and efficient document retrieval. Other libraries and repositories are indexed with other classification systems, such as ICD-10 (ICD), the 10th revision of the World Health Organization's (WHO) International Statistical Classification of Diseases and Related Health Problems. It contains codes for diseases, signs and symptoms, abnormal findings, complaints, social circumstances, and external causes of injury or diseases. Another classification system used for indexing document corpuses is SNOMED CT, a systematically organized collection of medical terms providing codes, terms, synonyms, and definitions used in clinical documentation and reporting. SNOMED CT classifies clinical findings, symptoms, diagnoses, procedures, body structures, organisms, and other etiologies, substances, pharmaceuticals, devices, and specimens.

Some systems might use machine learning, which uses processes and statistical models that computer systems can use to perform a specific task without requiring explicit instructions, instead perhaps relying on pattern matching and inference. More probably, artificial intelligence (AI) might be used. Machine learning processes can be used to build a mathematical model of sample or training data, in order to make predictions or decisions about input data without being explicitly programmed to perform the task.

An HCP might interact with the recommender system by submitting one or more EHRs that contain information about the HCP's patients. Data about conditions, therapies, test results, medical history, demographics, etc. might be extracted from the EHRs, and can be used to formulate queries to one or more search engines for one or more document corpuses, so as to retrieve medical literature that is relevant to the data extracted from the EHRs. These automatically formulated search queries can be expressed as Boolean expressions of search terms (e.g., “risk” AND “perforation” AND (“artery” OR “vein)). These search terms might be expanded to include not just synonyms (words that have the same meaning, such as “error” and “mistake”), but also hypernyms (words that are related by generalization, such as “chair” and “furniture”), hyponyms (words that are related by specialization, such as “animal” and “horse”), and meronyms (words that have a part-whole relationship, such as “ankle” and “leg”).

In some embodiments, the HCP does not need to explicitly provide the EHR documents to interact with the recommender system. For example, the recommender system might periodically scan the HCP's EHR database for files relating to the HCP's recent or upcoming patient visits.

The recommender system's response comprises zero or more documents that are selected from the search hits returned by the one or more database search engines that it uses to retrieve documents from one or more document corpuses. The search hits might be in the form of copies of the documents of the search results, abstracts, metadata, links (such as Universal Resource Identifiers or Universal Resource Locators), references, or some other identifiers of the documents that the search engine deems to be responsive to the search-engine input. In some cases, only references are provided and the user may then use another service, such as a pay-per-document service, to obtain the actual documents of interest.

The above-described basic recommender system might be augmented in various ways, as described below. In any single embodiment, one or more of these augmentations described herein might be used and others not used.

In one augmentation, alternative therapies are included in the search-term expansions of the database search queries used by the recommender system. If a patient is already being treated for a particular disease with one therapy, then the HCP will likely be interested in articles and guidelines concerning the effectiveness of this therapy. There are typically many candidate therapies for any given disease, so the HCP might also be interested in alternative therapies to the therapy currently prescribed for a patient. This is especially true for therapies that the HCP has not typically prescribed and for new therapies that have only recently proven to be effective. The search queries described above might therefore be expanded to not only include synonyms, hypernyms, hyponyms, and meronyms as noted above, but also to include search terms relating to alternative therapies.

In another augmentation, comorbidities are included in the search-term expansions of the database search queries used by the recommender system. Special therapies or combinations of therapies are often indicated when a patient presents with comorbidities. In order to recommend the most relevant articles for a patient from the medical literature, medical conditions should therefore not necessarily be treated independently when search queries are formulated in the system described above, but instead combined in the hope of finding medical literature that directly addresses comorbidities. Thus the search queries described above might be expanded to not only include synonyms, hypernyms, hyponyms, and meronyms as noted above, but also to include search terms for conditions that are typically comorbid to a condition already in the search query.

In another augmentation, genomic data are included in the search-term expansions of the database search queries used by the recommender system. A patient might have a genomic profile that correlates weakly, approximately, or incompletely with a disease state, or with a likely response to a particular therapy. To recommend more relevant articles for a patient from the medical literature, the search queries described herein might be expanded to not only include synonyms, hypernyms, hyponyms, and meronyms as noted above, but also to include search terms for conditions that are probabilistically likely given a patient's genomic profile, and search terms for therapies that are probabilistically likely to be of benefit given a patient's genomic profile.

In yet another augmentation, contextual information from the HCP's practice might influence the search-term expansions of the database search queries used by the recommender system. If a patient has a disease that the HCP rarely encounters, the system should probably return different medical-literature recommendations than if the patient has a disease that the HCP encounters frequently. For example, in the former case the recommendation of current standard-of-care guidelines might be useful; whereas in the latter case, the recommendation of recent novel research publications might be better. If a patient is of a demographic that the HCP encounters rarely, then that demographic might be given more weight in formulating and expanding the search queries used by the recommender system. If a patient has a genomic profile that they HCP encounters rarely, then that genomic profile might be treated differently for the purpose of search-query formulation. The recommender system may take contextual information into account by using second-order logic in the system's rule-based formulation of database search queries, so that the rules used can factor in quantified concepts, such as top and bottom deciles of a given patient population according to some criteria, or the most and least popular therapies prescribed for patients in a given patient population, or the correlations of different disease diagnoses in a given patient population.

As described herein, medical data about conditions, therapies, test results, medical history, demographics, etc. might be extracted from patient EHRs and used to formulate search-engine queries for one or more document corpuses, so as to retrieve medical literature that is relevant, or deemed relevant, to the medical data extracted from the EHRs. One process for extracting medical data from the EHRs uses “word spotting” to look for isolated words and phrases in the EHRs that have medical significance. However, looking at words and phrases in isolation might miss much of the linguistic nuance in EHRs. A more holistic analysis of the EHRs could lead to better, more appropriate recommendations of medical literature, but accurately parsing and interpreting the free text in EHRs remains technically challenging. In particular, interpreting negative statements is difficult. For example, these three sentences are superficially similar, as they all mention a test for cancer, but only one is a definitively positive statement about the patient currently having cancer: “the patient tested positive for cancer at her last visit”; “we tested the patient for cancer, but the results were indeterminate”; “a test for cancer was performed in 2002, and the results came back negative.” It can also be difficult to determine if the mention of a diagnosis applies to the patient or to a relative. For example, these three sentences are also superficially similar, as they all mention a diagnosis of cancer, but only one is a positive statement about the patient himself having cancer (and even that statement has some ambiguity): “the patient has some recollection that his aunt had a positive diagnosis of cancer”; “the patient's caregiver thinks that he was previously diagnosed with cancer”; “the patient mentioned that he thinks he has a genetic predisposition to cancer.”

In yet another augmentation, approximate parsing of EHR free text can be performed to formulate better search-engine queries that retrieve candidate medical literature from one or more document corpuses. Instead of using simple word spotting to attempt to extract categorical facts from EHRs, the search engine may use a type of “shallow parsing” that takes as input the text of an EHR and outputs a structured data structure comprising ordered pairs of possible medical facts along with their probabilities of being accurate. Multiple probability-fact pairs for the same fact can be combined to derive more accurate composite probabilities. Thus, if several statements of high probability regarding a positive cancer diagnosis are made over time for the same patient in one or more EHR documents, the cumulative interpretation will be one of a highly likely cancer diagnosis, and it might be preferred to have only medical facts of high likelihood be included in the search-engine queries for finding recommended medical literature.

A search engine might typically retrieve many more articles for a given query than may be usefully presented to an HCP. In another augmentation, the recommender system prioritizes medical literature recommendations that are more relevant to the HCP's EHRs in order to filter and decide on which subset of retrieved articles to present. One way to do this is to compute pair-wise similarity scores for medical-literature documents and EHR documents, using information-retrieval metrics. For example, Jaccard Similarity is a simple but intuitive measure of similarity between two documents, corresponding to the proportion of the number of common words to the number of unique words in both documents. In one embodiment, the recommender system might prioritize for presentation to the HCP user those retrieved articles with highest Jaccard Similarity scores relative to the HCP's EHRs.

A common mode of failure for recommender systems and search engines is to present only the items that are at the top of the list of retrieved articles according to some metric, without regard for variety. As an analogy, even if a user likes cowboy westerns, the user may be bored if a movie streaming service recommends only westerns. In another augmentation, a better presentation strategy that balances article rating with variety of the whole set of recommendations might be more useful to HCPs. The system might also present articles from the medical literature that are in service of specific pedagogic goals. As an example, the system may present articles from the literature relating to the same disease or therapy at regular intervals, so that the HCP is reminded of the relevant issues for that disease or therapy over time.

In some variations explanation and visualization of presented articles is provided. One presentation method is to present recommendations as scrollable lists of document titles, abstracts, and links. This kind of presentation makes it difficult to appreciate how the different recommended documents relate to each other.

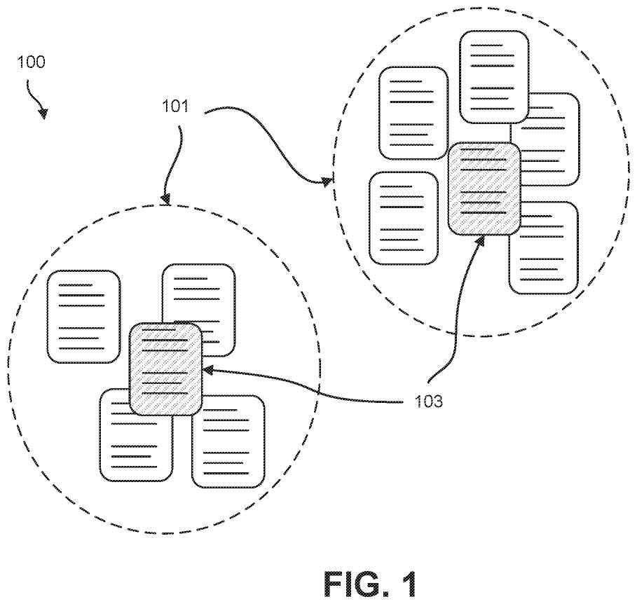

FIG. 1 illustrates a document-clustering visualization (100) that uses document clusters (101) and the visual highlighting of the most representative documents (103) for each cluster: this kind of visualization makes it easier for an HCP to comprehend the set of recommended documents being presented by the recommendation system. The system may also explain why each publication is being recommended to the HCP user, e.g., to which patient the publication relates, which disease or treatment it concerns, etc.

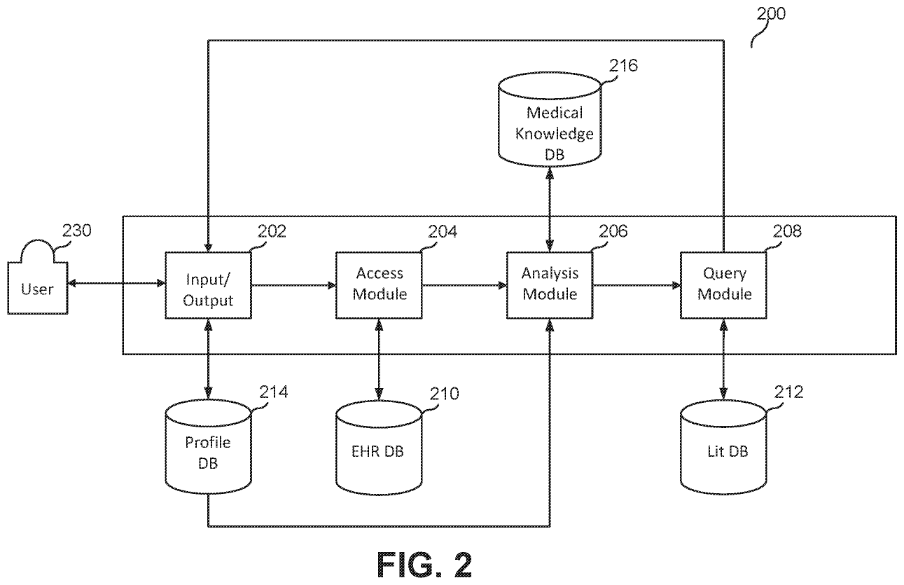

Referring now to FIG. 2, an exemplary system (200) for recommending, retrieving, and presenting medical literature of relevance to the patients of a healthcare professional (HCP) (230) has four modules: an input/output (I/O) module (202) configured to communicate with the HCP (230), an access module (204) configured to access a database of electronic health records (EHRs) (210) associated with the HCP's patients, an analysis module (206) configured to analyze the contents of the EHRs retrieved by the access module (204), and a medical-literature query module (208) configured to extract one or more publications from a medical-literature database (212) based on data provided by the analysis module (206).

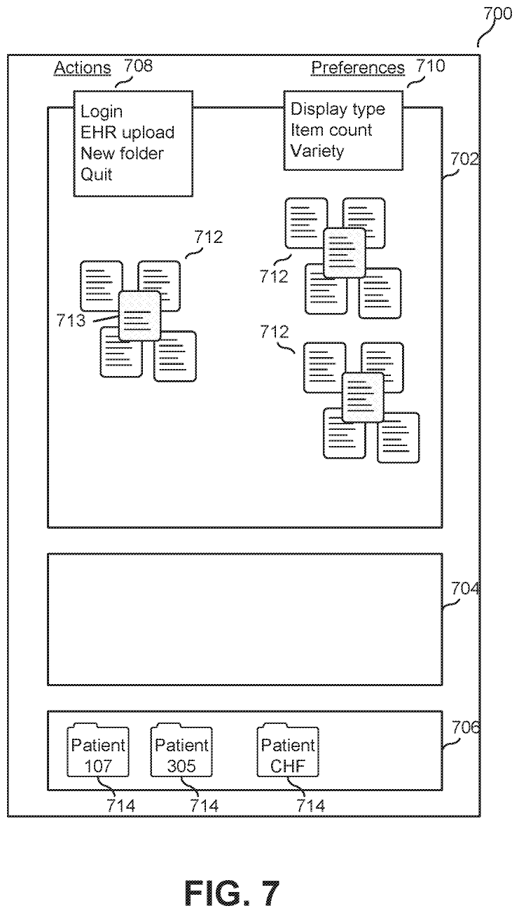

As shown in FIG. 2, an input/output (I/O) module (202) is configured to facilitate communication between the system (200) and an HCP user (230) via a shared user interface that is illustrated in FIGS. 7-9. With reference to FIG. 7, various input and output actions can be performed in the user interface (700). The “Actions” pulldown menu (708) has options for user login, the upload of EHR files, the creation of a new folder for storing medical-literature documents, and for user logout. The “Preferences” pulldown menu (710) has options for changing the display type, specifying the number of medical-literature items to be displayed, and for influencing the variety of the items displayed.

Icons representing medical-literature items are shown in the document display window (702). The system can use any one of several well-known document-clustering algorithms (714) to position icons for similar articles in visual clusters (712). For each cluster, the most representative articles (713), which are the ones with the greatest aggregate similarity scores for the documents in their respective clusters, may be highlighted. The contents of individual literature items can be examined in the text display window (704). Literature items can be organized by patient and stored in the folders presented in the folder window (706).

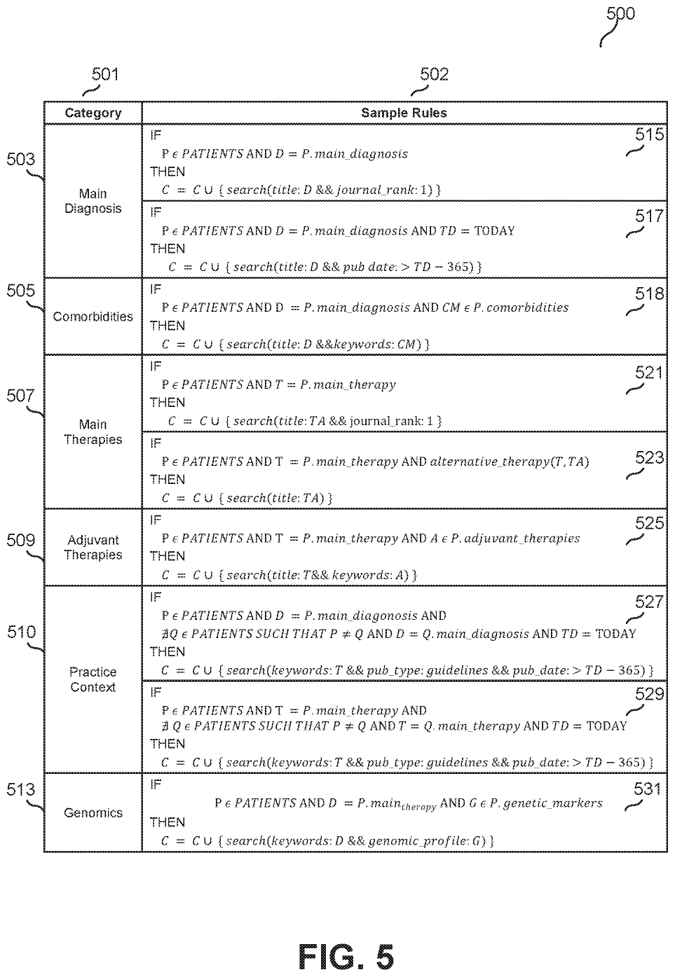

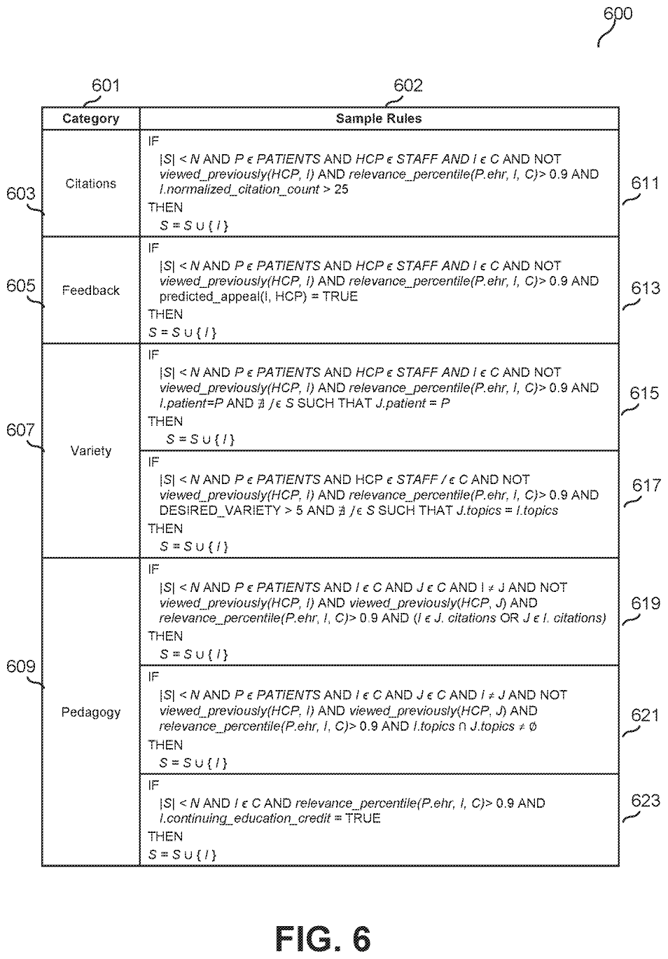

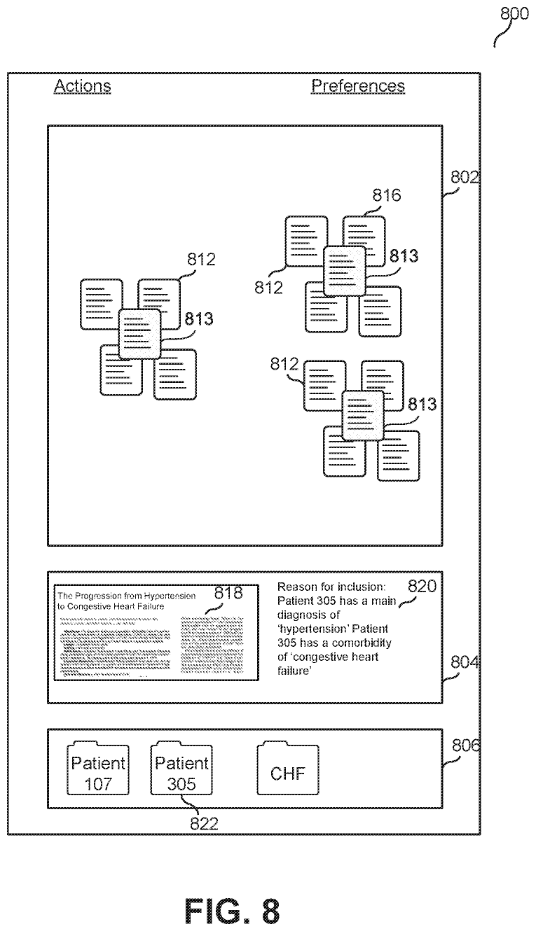

In an embodiment, the user can select document icons in the document display window (702), e.g., via left clicking with a mouse. FIG. 8 shows an interface 800 after item (816), (812), (813) has been selected. Once an icon is selected (802), the text (818) of the corresponding publication is shown in the text display window (804), which is scrollable and zoomable. In addition, the reason (820) for presenting the item to the user is shown in the window (804). This reason is determined automatically according to which expert-system rules (e.g., 500 of FIG. 5, 600 of FIG. 6) were used to select this item. The user can also store a literature item (806) in a folder (822) by selecting and dragging the document icon. Either the document-viewing action or the document-storing action might trigger the acquisition of a continuing-education credit for the item (816) if one is available.

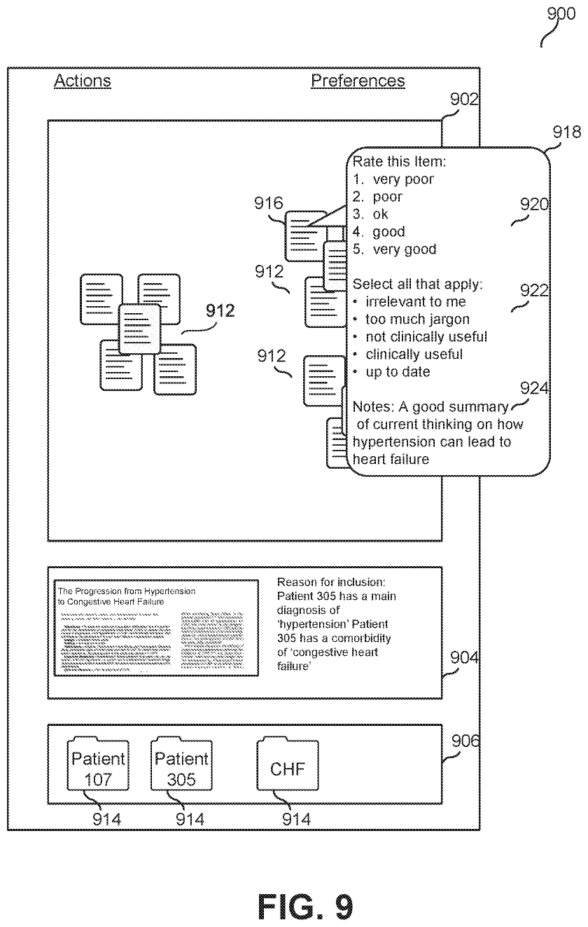

The user can also provide feedback and annotations for literature items, as shown in FIG. 9. When the user right clicks an icon in the display window (902), a user-feedback dialog (918) appears in the window. In this dialog, the user can provide a quantitative rating (920), qualitative notes (922), or free-text notes (924). These ratings and notes are stored in the user-profile database (214), from which they can be accessed and used by the I/O module (202) to determine which recommended articles to present to the HCP user (230).

Alternative display types, interaction widgets, and visual design elements with similar functionalities to those described above may be incorporated into the user interface (900).

Referring again to FIG. 2, an access module (204) can be configured to access a relational database of EHRs (210) so as to enable the retrieval of EHR files associated with a set of patients by an HCP (230). A relational database is a set of formally described tables from which data can be accessed. The standard user and application programming interface (API) of relational databases is the Structured Query Language (SQL). The access module (204) uses SQL commands inputted by the HCP user (230) to retrieve EHRs indexed by patient id, HCP id, date, location, etc.

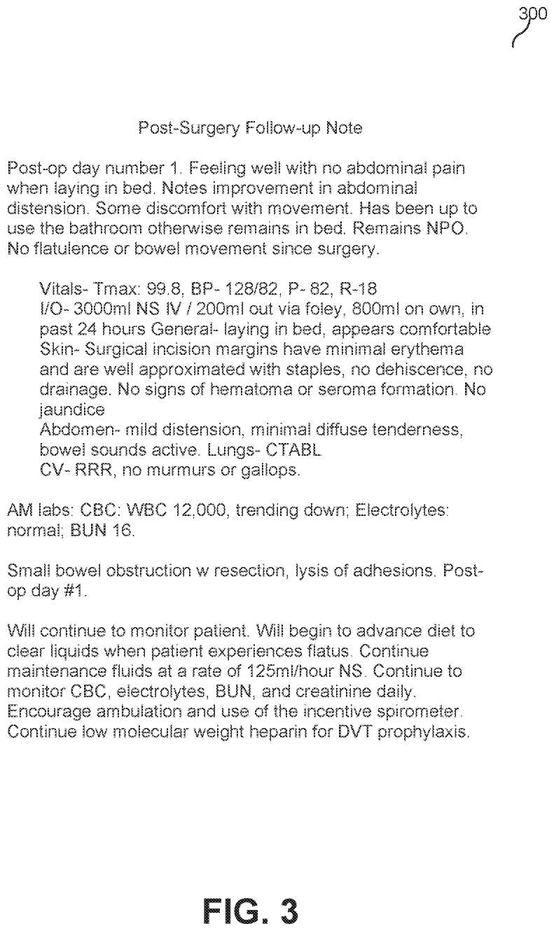

In an ideal world, the set of EHRs retrieved by the EHR access module (204) for each patient is complete, and the documents contain all important medical diagnosis and treatment facts as structured data, i.e., the facts are represented as data values of known data types in well-delineated document fields. The EHR analysis module (206) can then use standard database access methods to extract diagnosis and treatment facts from the structured portions of these EHRs. However, to get a more complete understanding of a patient's diagnoses and treatments likely requires analysis of the free-text notes in the EHR—see FIG. 3 for a typical free-text SOAP (subjective, objective, assessment, and plan) note that might be included in a patient's EHR (300). In such a scenario, the EHR analysis module (206) has to extract fragmentary evidence about diagnosis and treatment facts from text files and/or from the free-text notes in a potentially incomplete set of EHRs.

The difficulties associated with extracting diagnosis and treatment facts from free-text notes are illustrated by the following three sentences:

```
“Family History: the patient thinks that his mother might have died
  from colon cancer.”
“A colonoscopy performed in April of last year was negative for
  colorectal cancer.”
“After surgery I discussed various chemotherapy options as adjuvant
  therapy for the patient′s colorectal cancer.”
```

In an attempt to extract a diagnosis fact relating to colorectal cancer, one might try searching for the strings “colon” and “cancer” or for the strings “colorectal” and “cancer” in all three sentences. This is the kind of approach used in web-search engines: for example, the search string for conducting this search with a web search engine would be “(colon cancer) or (colorectal cancer).” However, a naive search like this will be successful for all three sentences, whereas only one of them indicates a positive diagnosis of colorectal cancer. Alternatively, various natural-language-processing (NLP) techniques might be used to parse and interpret the meanings of all three sentences. For example, a sophisticated NLP parser could yield the following parse of the second sentence above in terms of a hierarchical decomposition of noun phrases (NP), verb phrases (VP), prepositional phrases (PP), and adjectival phrases (ADJP):

```
(S (NP (NP (NP A colonoscopy)
      (VP performed
          (PP in
              (NP April))))
    (PP of
      (NP last year)))
  (VP was
    (ADJP negative
        (PP for
            (NP colorectal cancer)))))
```

This parse can then be further processed to extract logical statements about conditions and therapies. However, the cost and effort of developing grammars or other language models that can correctly process the often telegraphic and idiosyncratic notes in an EHR would be excessive.

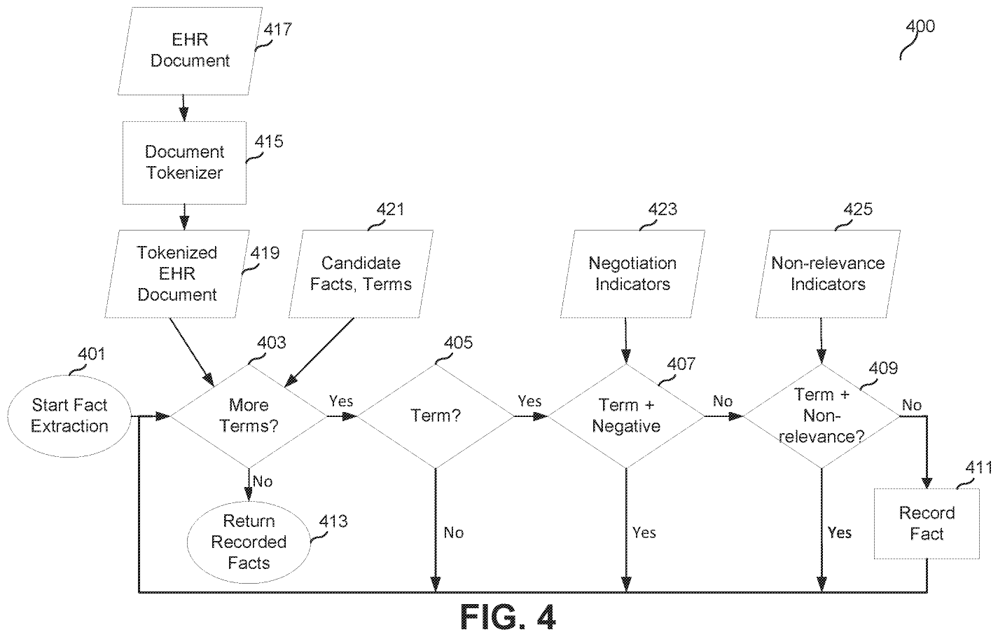

In some embodiments, an approach might be used that is intermediate between simple string searching and full NLP. In an embodiment, the system augments simple string searching with synonym substitution, so that a search for “colon” will also trigger a search for “colorectal,” for example. Additionally, the system may augment simple string searching with the concept of proximity in the document, allowing a search for strings that are nearby in a document. This comprises a particular form of “shallow parsing.” Specifically, FIG. 4 is a flowchart (400) that illustrates the shallow parsing in an example embodiment of the EHR analysis module (206), configured to extract diagnosis and treatment facts from the EHR for an individual patient, for a subset of patients in the HCP's practice (e.g., the patients that the HCP will see today or this week), or for all of the patients in the HCP's practice. The module takes as input EHR documents (417) for the patient that are tokenized (415) to break the documents into paragraphs, sentences, and words, resulting in tokenized EHR documents (419).

Next the EHR analysis module (206) extracts from a medical-knowledge database (216) a list of candidate facts (421) that are stored as ordered triples: (fact, relationship, term). For example, a fact might be “myocardial infarction,” a relationship might be “synonym,” and a term might be “heart attack.” In another ordered triple the fact “heart valve” might be related to the term “heart” by the relationship “meronym.” The set of associated terms for a given fact in aggregate constitutes the set of its synonyms, hypernyms, hyponyms, meronyms, and other related terms. These candidate-fact triples might be derived from standard data sources, such as the Unified Medical Language System (UMLS), a product of the US National Institute of Health's National Library of Medicine (https://www.nlm.nih.gov/research/umls/index.html). Given a comprehensive set of medical facts, the set of associated terms will be a comprehensive vocabulary of words and phrases for which to search in EHR documents.

Starting at step (401), the EHR analysis module (206) at step (403) iterates through the medical terms from the list of candidate facts (421), searching for these terms in the tokenized EHR document (419). This step (403) is performed by a “while loop” iteration: while there are still candidate terms from the list of candidate facts (421) to consider, the module selects a next term (405) and searches for it in the tokenized EHR document (419). If a term is found successfully at step (405), then it is passed on to the negation (407) and non-relevance (409) tests.

For the negation test, a term is combined with each of several negation indicators (423) and the combinations of term plus negation indicators are searched for in the tokenized EHR documents (419). For example, the term “heart attack” might be combined with the negation indicator “no evidence,” and if these two phrases are found in close proximity in the tokenized EHR documents (419), then the term “heart attack” is dropped from consideration for the document.

If a term passes this negation test, it is then combined with each of several non-relevance indicators (425) and the combinations of term plus non-relevance indicators are searched for in the tokenized EHR documents (419). For example, the term “heart attack” might be combined with the non-relevance indicator “family history,” and if these two phrases are found in close proximity in the tokenized EHR documents (419) then it is likely that this is part of the patient's family history and not relevant to the patient's present condition, so the term “heart attack” is dropped from consideration for the document.

If a term is found in a tokenized EHR document (405) and the term passes the negation test (407) and the non-relevance test (409), then all its corresponding facts from the list of candidate medical facts (421) are recorded (411). And when all terms have been considered and no more terms are found (403), all of the facts recorded at step (411) are returned (413) by the EHR analysis module (206).

Additional bookkeeping may be incorporated into the EHR analysis module (206) to record various metadata associated with the extracted diagnosis and treatment facts (e.g., dates of diagnosis, dates of treatment, patient ids, patient demographics, patient preferences, etc.) and to remove personal identification data (PID) from the extracted facts.

In artificial intelligence, an expert or rule-based system is a computer system that emulates human-expert decision making. Expert systems represent knowledge and actions explicitly as if-then rules: if a condition holds, then an action is taken. An inference module selects which rules to apply and in which order. Referring again to FIG. 2, a medical-literature query module (208) is configured to use expert-system methods to automatically formulate search queries that will retrieve a selection of medical-literature publications (e.g., journal articles, guidelines, presentations, videos, podcasts, blog postings, websites, etc.) from a medical-literature database (212). The automatically formulated search queries retrieve a selection of medical-literature publications that relate to the diagnosis and treatment facts extracted by the EHR analysis module (206). These diagnosis and treatment facts might relate to an individual patient, to a subset of patients in the HCP's practice (e.g., the patients that the HCP will see today or this week), or to all of the patients in the HCP's practice. The automatically formulated search queries might also use the metadata associated with the extracted diagnosis and treatment facts to further refine the selection of medical-literature publications (e.g., by prioritizing recent diagnoses and treatments, or by prioritizing patients with high-risk conditions, etc.). The automatically formulated search queries might also reflect a patient's comorbidities and adjuvant therapies (rather than consider each diagnosis and treatment in isolation), as well as a patient's genomic profile in its entirety (rather than consider individual genomic markers in isolation). In an embodiment, the medical-literature query module (208) is implemented via a logic-programming language (as an example, the Prolog language) that supports dynamic assertion and manipulation of facts (e.g., to record user feedback), second-order predicates that permit logical statements over all EHR documents (e.g., setof and bagof in Prolog), and the ability to interface with external modules (e.g., to use ML libraries written in other languages).

The rules in the expert system of the query module (208) fall into two general categories and several subcategories. FIG. 5 contains the general category of expert-system rules (500) that are used to formulate search queries in the query module (208). The subcategories of the query-formulation rules are listed in the chart's left column (501), and sample rules for each subcategory are shown in the chart's right column (502). FIG. 6 contains the general category of expert-system rules that decide which of the retrieved items from the medical literature to present to the user (600), and which are used in the I/O module (202). The subcategories of the presentation rules are listed in the chart's left column (601), and sample rules for each subcategory are shown in the chart's right column (602). The sample rules presented in FIG. 5 and FIG. 6 are written in pseudocode for ease of understanding. These pseudocode rules may be reduced to executable code in a programming language designed to support expert systems, an example of which is Prolog. The application of the query-formulation rules (500) may occur before the application of the presentation rules (600). The sample rules presented in FIG. 5 and FIG. 6 are listed in arbitrary order, but there may be advantages to ordering the application of the query-formulation rules (500) and to ordering the application of the presentation rules (600). For example, all of the presentation rules 600) in FIG. 6 collect articles for presentation to the user until a threshold number of articles is collected; rules that fire before others therefore are more likely to contribute to the presented set of articles. The sample rules presented in FIG. 6 also assume access to the data in the EHR database (210), such as a patient's diagnosis, therapies, and genetic markers, for a set of patients. The sample rules presented in FIG. 6 further assume the ability to invoke searches of a relational database of medical-literature publications (212) with fields like title, keywords, pub_date, etc. For example, the MEDLINE database of references and abstracts for the life sciences and biomedical topics is one such database that can be accessed through the PubMed search engine and equivalent systems.

Returning to the query-formulation rules (500) in FIG. 5 that are used by the query module (208), the first subcategory of query-formulation rules (503) relates to a patient's main diagnosis. One sample rule (515) causes a search of the medical-literature database for publications that include the patient's main diagnosis in the title field and that have a top ranking in the journal_rank field. Another sample rule (517) invokes a search of the medical-literature database for publications that include the patient's main diagnosis in the title field and that have been published within the past 365 days. Publications resulting from these and other searches described in (500) are collected in the set C of candidate medical-literature items.

The second subcategory of query-formulation rules (505) relates to a patient's comorbidities. Typical comorbidities for a given condition may be stored as ordered pairs (condition, comorbidity) in a medical-knowledge database (216). The logical predicate comorbid(condition, comorbidity) retrieves and matches successive values for the comorbidity variable, given an instantiated value for the condition variable. One sample rule (518) of the comorbidity category 505 calls the comorbidities( ) predicate in the course of causing a search of the medical-literature database for publications that include the patient's main diagnosis in the title field and that has one of the patient's comorbidities in the keywords field.

The third subcategory of query-formulation rules (507) relates to a patient's main therapy. One sample rule (521) causes a search of the medical-literature database for publications that include the patient's main therapy in the title field and that has a top ranking in the journal_rank field. Another sample rule (523) concerns alternative therapies. Typical alternative therapies for a given therapy may be stored as ordered pairs (therapy, alt_therapy) in a medical-knowledge database (216). The logical predicate alternative_therapy(therapy, alt_therapy) retrieves and matches successive values for the alt_therapy variable, given an instantiated value for the therapy variable. The sample rule (523) calls the alternative_therapy( ) predicate in the course of causing a search of the medical-literature database for publications that include alternative therapies to a patient's main therapy in the title field.

The fourth subcategory of query-formulation rules (509) relates to a patient's adjuvant therapies. One sample rule (525) causes a search of the medical-literature database for publications that include the patient's main therapy in the title field and that has one of the patient's adjuvant therapies in the keywords field.

The fifth subcategory of query-formulation rules (510) considers the diagnoses and therapies of a single patient in the context of the diagnoses and therapies of all the patients in the consideration set, which typically might be all the patients in ‘san HCP's practice, or all of the patients with recent or upcoming appointments. One sample rule (527) causes a search of the medical-literature database for recently published guidelines concerning diagnoses that are unique to one patient in the consideration set. Another sample rule (529) causes a search of the medical-literature database for recently published guidelines concerning therapies that are unique to one patient in the consideration set. Note that both of these sample rules involve quantification over sets, namely the sets of patients that have a particular main diagnosis or main therapy. Quantification over sets is a feature of second-order logic, so it is essential that the programming language used to implement our system be capable of supporting higher-order logic. Additional contextual rules might relate to demographics: if a patient is of a demographic that the HCP encounters rarely, then that demographic might be included in the search terms of a query when it might not otherwise be included.

The sixth subcategory of query-formulation rules (513) relates to a patient's genomic profile. One sample rule (531) causes a search of the medical-literature database for publications that include the patient's main diagnosis in the title field and that has at least one of the patient's genetic markers in the genomic profile field.

Turning now to the presentation rules (600) in FIG. 6 that are used in the I/O module (202), the purpose of these rules is to select a subset of the retrieved medical-literature publications for presentation to the HCP, optimizing for publication quality, relevance, and variety, and also for pedagogical effect. It is important to note that not all of the retrieved publications returned by the query module (208) will be highly relevant to the HCP's patients. For example, a retrieved publication that is responsive to the keyword of “dementia” might address the wrong demographic (e.g., the retrieved publication might be about early onset of dementia in women, whereas the relevant HCP's patient might be an elderly man with dementia). Tailoring the query-formulation rules (600) to retrieve only highly relevant publications may, in practice, yield an unwieldy number and scope of query-formulation rules. Another approach is to allow the query-formulation rules (600) to cast a wide net by limiting their specificity and then to filter the retrieved publications for relevance to the HCP's patients. The system may incorporate relevance filtering via the relevance_percentile(T, I, C) predicate that is included in each of the presentation rules (600): this predicate considers all the candidate publication items C retrieved by the query module (208) and ranks them by relevance to the text file document T, and then returns the rank of the particular publication item I in that sorted order. When the text file T is the EHR document for a patient, this predicate gives a rank measure of relevance to patient P for publication item I from the candidate set of publication items C. The relevance_percentile(T, I, C) predicate may compute a score using a document-similarity metric in which stop words are removed, word stemming is applied, and then common terms in the two documents are counted after they have been weighted by term frequency within the documents, and inversely weighted by term frequency in a representative corpus of the medical literature. In addition, the document-similarity approach can be augmented by the artificial inclusion in the document of words and phrases like “therapy,” “treatment,” “drug trial,” “review,” “study,” and “guideline.” Furthermore, demographic appropriateness can be reflected in the relevance score by the inclusion of synonyms relating to the patient's demographic data, e.g., if there is mention in a document that the patient is “80 years old” then adding the words “elderly” and “geriatric” to the document might increase the relevance score for publications concerning older patients. Note that the relevance_percentile(T, I, C) predicate involves quantification over a set, namely the set of candidate publication items C. As quantification over sets is a feature of second-order logic, it is advantageous that the programming language used to be capable of supporting higher-order logic.

Using the scoring publication relevance via the relevanc_epercentile(T, I, C) predicate, the system may apply presentation rules (600). The first subcategory (603) of presentation rules relates to the citation counts for the retrieved medical-literature publications in the set S of candidate medical-literature items. One sample rule (611) in the citation category (603) states that if the target number N of publications in the presentation set S has not been met, a published item I has not been viewed by the user HCP (i.e., if it meets the criterion of novelty), the published item I is in the 90th percentile or higher of relevance for the patient P (i.e., if it meets the criterion of relevance) relative to the candidate set of publication items C, and the published item I has a normalized citation count of more than 25 citations per year, then it should be added to the presentation set S. For very recent publications, a normalized citation count can be estimated using Machine Learning (ML) based on the content of the publication, e.g., its keywords, authors, authors' affiliations, references, etc. Supervised ML may be used for this estimation task, based on a training set comprising non-recent publications and their normalized citation counts.

The second subcategory (605) of query-formulation rules relates to user feedback. One sample rule (613) adds an item I to the presentation set S if item I meets the usual criteria of novelty and relevance and if item I is predicted to appeal to the user HCP. The predicate predicted appeal(I, H) returns a score indicating how likely the item I is to be of interest to the HCP H, which in one embodiment can be computed using Machine Learning (ML). Supervised ML may be used for this predictive task, based on a training set comprising publications and their ratings by users who have viewed them, and on the ratings for an individual HCP stored in the individual's user-profile database (214).

The third subcategory (607) of presentation rules ensures that the publication items in the presentation set S will exhibit sufficient variety. One sample rule (615) adds an item I to the set S if item I meets the usual criteria of novelty and relevance and if it is the first item in S to concern patient P. Another sample rule (617) adds an item I to the set S if item I meets the usual criteria of novelty and relevance, and if the level of variety specified in the I/O module pulldown menu (710) is sufficiently high and the item I is the first item in S to concern a particular topic. Note that both of these sample rules involve further quantification over a set, namely the set of items in S. As noted previously, quantification over sets is a feature of second-order logic, so it is essential that the programming language used to implement our system be capable of supporting higher-order logic.

The fourth subcategory of presentation rules (609) concern education and pedagogy. For example, reinforcing previously viewed publications is a useful educational strategy. So one sample rule (619) adds an item I to the set S if item I meets the usual criteria of novelty and relevance and if there is a citing relationship between items I and J and item J was previously viewed by the user HCP. Another sample rule (621) adds an item I to the set S if item I meets the usual criteria of novelty and relevance and if item I is topically similar to item J and item J was previously viewed by the user HCP. Another sample rule (623) adds an item I to the set S if item I meets the usual criteria of novelty and relevance and if item I is eligible for some form of continuing-education credit.

Additional rules may be added to the various categories and subcategories listed in (500) and (600), and constant numeric parameters may be modified advantageously, e.g., the normalized citation count in sample rule (611).

According to one embodiment, the techniques described herein are implemented by one or generalized computing systems programmed to perform the techniques pursuant to program instructions in firmware, memory, other storage, or a combination. Special-purpose computing devices may be used, such as desktop computer systems, portable computer systems, handheld devices, networking devices or any other device that incorporates hard-wired and/or program logic to implement the techniques.

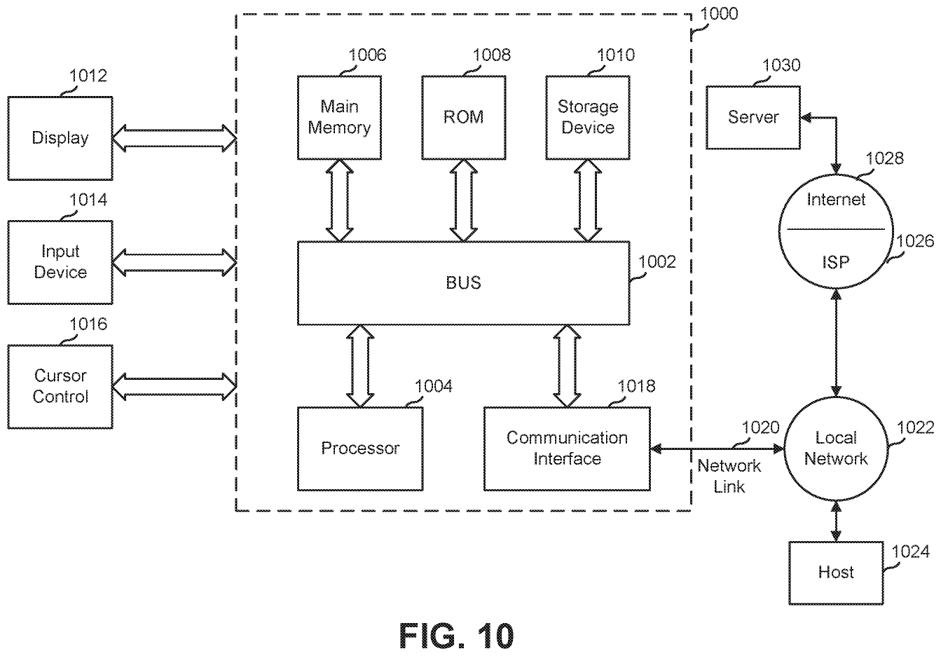

For example, FIG. 10 is a block diagram that illustrates a computer system (1000) upon which an embodiment may be implemented. Computer system (1000) includes a bus (1002) or other communication mechanism for communicating information, and a processor (1004) coupled with bus (1002) for processing information. Processor (1004) may be, for example, a general purpose microprocessor.

Computer system (1000) also includes a main memory (1006), such as a random access memory (RAM) or other dynamic storage device, coupled to bus (1002) for storing information and instructions to be executed by processor (1004). Main memory (1006) also may be used for storing temporary variables or other intermediate information during execution of instructions to be executed by processor (1004). Such instructions, when stored in non-transitory storage media accessible to processor (1004), render computer system (1000) into a special-purpose machine that is customized to perform the operations specified in the instructions.

Computer system (1000) further includes a read only memory (ROM) (1008) or other static storage device coupled to bus (1002) for storing static information and instructions for processor (1004). A storage device (1010), such as a magnetic disk or optical disk, is provided and coupled to bus (1002) for storing information and instructions.

Computer system (1000) may be coupled via bus (1002) to a display (1012), such as a computer monitor, for displaying information to a computer user. An input device (1014), including alphanumeric and other keys, is coupled to bus (1002) for communicating information and command selections to processor (1004). Another type of user input device is cursor control (1016), such as a mouse, a trackball, or cursor direction keys for communicating direction information and command selections to processor (1004) and for controlling cursor movement on display (1012). This input device typically has two degrees of freedom in two axes, a first axis (e.g., x) and a second axis (e.g., y), that allows the device to specify positions in a plane.

Computer system (1000) may implement the techniques described herein using customized hard-wired logic, one or more ASICs or FPGAs, firmware and/or program logic which in combination with the computer system causes or programs computer system (1000) to be a special-purpose machine. According to one embodiment, the techniques herein are performed by computer system (1000) in response to processor (1004) executing one or more sequences of one or more instructions contained in main memory (1006). Such instructions may be read into main memory (1006) from another storage medium, such as storage device (1010). Execution of the sequences of instructions contained in main memory (1006) causes processor (1004) to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical or magnetic disks, such as storage device (1010). Volatile media includes dynamic memory, such as main memory (1006). Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, an EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus (1002). Transmission media can also take the form of acoustic or light waves, such as those generated during radio-wave and infra-red data communications.

Various forms of media may be involved in carrying one or more sequences of one or more instructions to processor (1004) for execution. For example, the instructions may initially be carried on a magnetic disk or solid state drive of a remote computer. The remote computer can load the instructions into its dynamic memory and send the instructions over a network connection. A modem or network interface local to computer system (1000) can receive the data. Bus (1002) carries the data to main memory (1006), from which processor (1004) retrieves and executes the instructions. The instructions received by main memory (1006) may optionally be stored on storage device (1010) either before or after execution by processor (1004).

Computer system (1000) also includes a communication interface (1018) coupled to bus (1002). Communication interface (1018) provides a two-way data communication coupling to a network link (1020) that is connected to a local network (1022). For example, communication interface (1018) may be an integrated services digital network (ISDN) card, cable modem, satellite modem, or a modem to provide a data communication connection to a corresponding type of telephone line. Wireless links may also be implemented. In any such implementation, communication interface (1018) sends and receives electrical, electromagnetic or optical signals that carry digital data streams representing various types of information.

Network link (1020) typically provides data communication through one or more networks to other data devices. For example, network link (1020) may provide a connection through local network (1022) to a host computer (1024) or to data equipment operated by an Internet Service Provider (ISP) (1026). ISP (1026) in turn provides data communication services through the world wide packet data communication network now commonly referred to as the “Internet” (1028). Local network (1022) and Internet (1028) both use electrical, electromagnetic or optical signals that carry digital data streams. The signals through the various networks and the signals on network link (1020) and through communication interface (1018), which carry the digital data to and from computer system (1000), are example forms of transmission media.

Computer system (1000) can send messages and receive data, including program code, through the network(s), network link (1020) and communication interface (1018). In the Internet example, a server (1030) might transmit a requested code for an application program through Internet (1028), ISP (1026), local network (1022) and communication interface (1018). The received code may be executed by processor (1004) as it is received, and/or stored in storage device (1010), or other non-volatile storage for later execution.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory.

Conjunctive language, such as phrases of the form “at least one of A, B, and C,” or “at least one of A, B and C,” unless specifically stated otherwise or otherwise clearly contradicted by context, is otherwise understood with the context as used in general to present that an item, term, etc., may be either A or B or C, or any nonempty subset of the set of A and B and C. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present.

The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

Further embodiments can be envisioned to one of ordinary skill in the art after reading this disclosure. In other embodiments, combinations or sub-combinations of the above-disclosed invention can be advantageously made. The example arrangements of components are shown for purposes of illustration and it should be understood that combinations, additions, re-arrangements, and the like are contemplated in alternative embodiments of the present invention. Thus, while the invention has been described with respect to exemplary embodiments, one skilled in the art will recognize that numerous modifications are possible.

For example, the processes described herein may be implemented using hardware components, software components, and/or any combination thereof. The specification and drawings are, accordingly, to be regarded in an illustrative rather than a restrictive sense. It will, however, be evident that various modifications and changes may be made thereunto without departing from the broader spirit and scope of the invention as set forth in the claims and that the invention is intended to cover all modifications and equivalents within the scope of the following claims.

All references, including publications, patent applications, and patents, cited herein are hereby incorporated by reference to the same extent as if each reference were individually and specifically indicated to be incorporated by reference and were set forth in its entirety herein.

What is claimed is:

1. A system, comprising:
a network interface;
a computing system; and
at least one computing device configured to implement one or more services, wherein the one or more services are configured to:
access, over a network using the network interface, a set of electronic health record documents relating to a set of patients from at least a first data store;
use a first set of AI techniques to analyze the contents of the set of electronic health record documents to extract a set of extracted medical facts;
use a second set of AI techniques to formulate a set of database queries based on the set of extracted medical facts that are used to retrieve, over a network using the network interface, a set of resource locators, each resource locator for a retrieved medical-literature publication from at least a second data store that are relevant to the set of extracted medical facts;
use a third set of AI techniques to determine a subset of the set of resource locators to present to a user; and
present the subset of the set of the resource locators to the user via the computing system's display.

2. The system of claim 1, in which the first set of AI techniques analyzes the contents of the set of electronic health record documents to extract a set of extracted medical facts using natural language processing.

3. The system of claim 1, in which the second set of AI techniques used to formulate a set of database queries based on the set of medical facts that are used to retrieve, over a network using the network interface, a set of retrieved medical-literature publications from the second data store that are relevant to the set of extracted medical facts, the system comprising one or more of:
expert-system rules that consider the diagnoses of one or more patients in the set of patients;
expert-system rules that consider the therapies of one or more patients in the set of patients;
expert-system rules that consider the comorbidities of one or more patients in the set of patients;
expert-system rules that consider adjuvant or alternative therapies for one or more patients in the set of patients; and
expert-system rules that consider the genomic profiles of one or more patients in the set of patients.

4. The system of claim 1, in which the third set of AI techniques used to determine which subset of the set of retrieved medical-literature publications to present to a user and how to present them, comprise one or more of these methods:
term-matching and term-weighting methods to rate relevance of publications in the set of retrieved medical-literature publications to present to a user;
expert-system rules that consider the citation counts of publications in the set of retrieved medical-literature publications to present to a user;
expert-system rules that consider user-supplied feedback regarding the publications in the set of retrieved medical-literature publications to present to a user;
expert-system rules that promote variety in the subset of the set of retrieved medical-literature publications to present to a user;
expert-system rules that apply pedagogical strategies in the selection of the subset of the set of retrieved medical-literature publications to present to a user;
the provision of automatically generated explanations associated with the subset of the set of retrieved medical-literature publications to present to a user; and
document-clustering methods for presenting the subset of the set of retrieved medical-literature publications to a user.

5. A computer-implemented method for querying at least one document database, comprising:
under the control of one or more computer systems configured with executable instructions:
1) receiving a specified electronic health record from the HCP via a user interface;
2) retrieving a first plurality of documents from at least one document database based on the specified electronic health record;
3) applying a rule to the first plurality of documents that embodies a presentation or pedagogical strategy to remove at least one document from the plurality of documents to produce a second plurality of documents; and
4) presenting the second plurality of documents to the HCP via the user interface.

6. The method of claim 5, wherein retrieving the first plurality of documents from the at least one document database further comprises:
identifying at least one therapy term in the specified electronic health record;
querying a medical knowledge database with the therapy term to identify an alternative therapy search term;
querying the at least one document database with the alternative therapy search term to identify a plurality of alternative therapy documents; and
including the alternative therapy documents in the first plurality of documents.

7. The method of claim 5, wherein retrieving the first plurality of documents from the at least one document database further comprises:
identifying at least two conditions in the specified electronic health record;
querying a medical knowledge database with the at least two conditions to identify a comorbidity search term;
querying the at least one document database with the comorbidity search term to identify a plurality of comorbidity documents; and
including the comorbidity documents in the first plurality of documents.

8. The method of claim 5, wherein retrieving the first plurality of documents from the at least one document database further comprises:
identifying at least one genomic profile in the specified electronic health record;
querying a medical knowledge database with the genomic profile to identify at least one genomic search term;
querying the at least one document database with the at least one genomic search term to produce a plurality of genomic documents; and
including the genomic documents in the first plurality of documents.

9. The method of claim 5, wherein applying a rule to the first plurality of documents that embodies a presentation or pedagogical strategy to remove at least one document from the plurality of documents to produce a second plurality of documents further comprises identifying the documents in the first plurality of documents that are most relevant to the specified electronic health record received from an HCP.

10. A computer-implemented method for querying at least one document database, comprising:
under the control of one or more computer systems configured with executable instructions:
1) assembling contextual information about an HCP's practice by processing a plurality of electronic health records associated with an HCP;
2) receiving a specified electronic health record from the HCP via a user interface;
3) retrieving a first plurality of documents from at least one document database based on the specified electronic health record;
4) applying a rule to the first plurality of documents based on the contextual information about an HCP's practice to remove at least one document from the plurality of documents to produce a second plurality of documents; and
5) presenting the second plurality of documents to the HCP via the user interface.

11. The method of claim 10, wherein assembling contextual information about an HCP's practice includes:
determining the frequency of a condition in the plurality of electronic health records.

12. The method of claim 10, wherein assembling contextual information about an HCP's practice includes:
determining the frequency of a therapy in the plurality of electronic health records.

13. The method of claim 10, wherein applying a rule to the first plurality of documents based on the contextual information about an HCP's practice further comprises:
identifying the documents in the first plurality of documents that are most relevant to the specified electronic health record received from an HCP.
